(19) United States
(12) Patent Application Publication
(10) Pub. No.: US 2012/0128593 A1
(43) Pub. Date: May 24, 2012

(54) USE OF A MAGNETIC RESONANCE IMAGING MEDIUM COMPRISING HYPERPOLARIZED 13C PYRUVATE FOR THE DETECTION OF INFLAMMATION OR INFECTION

(76) Inventors: Yi-Fen YEN, Menlo Park, CA (US); John D. Mackenzie, Palo Alto, CA (US); Dirk Mayer, Redwood City, CA (US); Daniel M. Spielman, Menlo Park, CA (US)

(21) Appl. No.: 13/248,247

(22) Filed: Sep. 29, 2011

Related U.S. Application Data
(63) Continuation of application No. PCT/EP2010/005391, filed on Mar. 25, 2010.

(51) Int. Cl. A61K 49/10; C07C 59/19; C12Q 1/02

(52) U.S. Cl. 424/9.3; 435/29; 562/577

(57) ABSTRACT

The invention relates to a method of ¹³C-MR imaging, ¹³C-MR spectroscopy and/or 13C-MR spectroscopic imaging of inflammation or infection using an imaging medium which comprises a hyperpolarized ¹³C-substance.

### CROSS REFERENCE TO RELATED APPLICATIONS

This application is a continuation application of commonly owned International Application No. PCT/EP2010/053912, filed 25 Mar. 2010. International Application No. PCT/EP2010/053912 claims priority to EP Patent Application 09157152.1 filed 2 Apr. 2009. Each of the foregoing applications are hereby incorporated by reference in its entirety.

### FIELD OF THE INVENTION

Embodiments of the invention relates to a method of carbon-13 (¹³C) magnetic resonance (MR) imaging or spectroscopy of inflammation or infection using an imaging medium comprising a hyperpolarized ¹³C-substance. Embodiments of the invention relates to the application of carbon-13 labelled molecules that have been hyperpolarized for subsequent imaging with MR imaging to detect or monitor inflammation or infection.

### BACKGROUND

Inflammation is the biological response to harmful agents that damage bodily tissues. Inflammation is a balancing act between host defenses and tissue injury. Key to the inflammatory response is the immune system and vascular tissues. The immune system is composed of white blood cells and molecules that help the body fight infection, remove noxious stimuli, and repair damaged tissues. During the inflammatory process the immune system and increased blood flow help clear pathogens and repair injured tissues.

Inflammation involves the recruitment of new blood vessels to bring nutrients and additional components of the immune system to the site of infection or injury. Although inflammation often is the result of an exogenous pathogen (e.g. bacteria, virus, fungus, parasite, prions, and viroids) other initiators of an inflammatory response include autoantigens, trauma, allergens, and irritants. In the absence of inflammation, wounds and infections would not heal and progressive destruction of the tissue would lead to demise of the organism. Inflammation often signals that an underlying disease is present as the body tries to rid the disease. An infection is the colonization of a host organism by a foreign species that often results in clinically evident disease. The foreign species is usually a microscopic pathogen such as a colony of bacteria, fungus, virus, parasite prion, or viroid. Inflammation is the mechanism mounted by the host organism to clear an infection. Inflammation may also occur to clear autoantigens, damaged tissue (e.g. trauma), allergens, or irritants.

However, inflammation can also lead to a host of problems when misregulated or left unchecked, including autoimmune diseases, allergies, atherosclerosis, inflammatory and degenerative arthritis, asthma, chronic bronchitis, chronic obstructive pulmonary disease (COPD), and multiple sclerosis. It is for this reason that inflammation is normally tightly regulated by the body. Inflammation can be classified as either acute or chronic. Acute inflammation is the initial response of the body to harmful stimuli and is achieved by the increased movement of plasma and white blood cells from the blood into the injured tissues. A cascade of biochemical events propagates and matures the inflammatory response, involving the local vascular system, the immune system, and various cells within the injured tissue. Prolonged inflammation, known as chronic inflammation, leads to a progressive shift in the type of cells which are present at the site of inflammation and is characterised by simultaneous destruction and healing of the tissue from the inflammatory process.

Inflammatory and infectious diseases share similar mechanisms on the molecular and cellular level. These diseases result in activation of the immune system, and are often difficult disease processes to clinically detect and monitor. Currently, the options for the imaging detection of inflammation and infection are limited, and no good clinical test exists for detecting and monitoring the response of these diseases to therapy. Clinicians must rely on subjective measures of how the patient feels, secondary signs such as blood tests (white blood cell count, CRP, etc), non-specific nuclear medicine imaging, or late anatomic changes of disease based on anatomic imaging (conventional MRI, ultrasound, computed tomography, and radiographs). As an example, rheumatoid arthritis is a common disease affecting ˜1% of the geriatric population, and currently, no good non-invasive test exists for detecting or monitoring rheumatoid arthritis. Clinicians are often left with subjective measures for diagnosing the disease and for determining how the patient is responding to treatment. Hence there is an interest in detecting inflammation and infection non-invasively in vivo in the human or non-human animal body.

MR detection such as MR imaging (MRI), MR spectroscopy (MRS) and MR spectroscopic imaging (MRSI) could be valuable tools for detecting inflammation and infection and these tools have become particularly attractive to physicians as they allow for obtaining images of a patient's body or parts thereof in a non-invasive way and without exposing the patient and the medical personnel to potentially harmful radiation such as x-rays. Because of its high quality images with excellent soft tissue contrast and good spatial and temporal resolution, MRI is the favourable imaging technique of soft tissue and organs.

### BRIEF SUMMARY

It has now been found that a hyperpolarized ¹³C-substance can be used as an agent for detecting inflammation and infection in the human or non-human animal body using ¹³C-MRI, ¹³C-MRS, or ¹³C-MRSI.

One embodiment of the present invention is directed to a method for detecting inflammation or infection by ¹³C-MR imaging, ¹³C-MR spectroscopy and/or ¹³C-MR spectroscopic imaging. An imaging medium comprising hyperpolarized ¹³C-pyruvate is used, and inflammation or infection are detected by high ¹³C-signal intensity from ¹³C-lactate or an increased rate of formation of ¹³C-lactate.

A further embodiment of the present invention is directed to use of hyperpolarized ¹³C-pyruvate for the manufacture of an imaging medium. The imaging medium is for use in a method for detecting inflammation or infection by ¹³C-MR imaging, ¹³C-MR spectroscopy and/or ¹³C-MR spectroscopic imaging. Inflammation or infection are detected by high ¹³C-signal intensity from ¹³C-lactate or an increased rate of formation of ¹³C-lactate.

A yet further embodiment of the present invention is directed to a method for detecting inflammation or infection in a human or non-human animal body by ¹³C-MR imaging, ¹³C-MR spectroscopy and/or ¹³C-MR spectroscopic imaging. An imaging medium comprising hyperpolarized ¹³C-pyruvate has been preadministered to the human or non-human animal body, and inflammation or infection are detected by high ¹³C-signal intensity from ¹³C-lactate or an increased rate of formation of ¹³C-lactate.

An even further embodiment of the present invention is directed to use of an imaging medium comprising hyperpolarized ¹³C-pyruvate. Such imaging medium is used in a method for detecting inflammation or infection in a human on non-human animal body of ¹³C-MR imaging, ¹³C-MR spectroscopy and/or ¹³C-MR spectroscopic imaging. Inflammation or infection are detected by high ¹³C-signal intensity from ¹³C-lactate or an increased rate of formation of ¹³C-lactate.

Other features and advantages of this invention will be better appreciated from the following detailed description.

### BRIEF DESCRIPTION OF THE DRAWINGS

Embodiments of the invention will now be described in greater detail with reference to the accompanying Figures.

FIG. 1 shows metabolic maps of arthritic joints.

FIG. 2 shows time resolved imaging relating to the production of [1-13C]lactate.

### DETAILED DESCRIPTION

Thus, in a first aspect embodiments of the invention provides a method of ¹³C-MR imaging and/or ¹³C-MR spectroscopy and/or ¹³C-MR spectroscopy imaging for detecting inflammation or infection using an imaging medium comprising a hyperpolarized ¹³C-substance. Such substances should contain nuclei with longitudinal relaxation time constants (T₁) that are greater than 10 seconds, preferably greater than 30 seconds and even more preferably greater that 60 seconds. Such so called “high T₁ agents” are for instance described in WO-A-99/35508. Alternatively, T₁ values of possible substances may be found in the literature or may be determined by acquiring an NMR spectrum of the possible substance, e.g. a ¹³C-NMR spectrum to determine the T₁ of a ¹³C-labelled possible substance.

Preferred hyperpolarized ¹³C-substances are biomolecules that play a role in the metabolic processes in the human and non-human animal body. Especially preferred substances are thus endogenous compounds, more preferably endogenous compounds that play a role in a metabolic process in the human or non-human animal body. Especially preferred substances are selected from amino acids (in protonated or deprotonated form), preferably alanine, glycine, glutamine, glutamic acid, cysteine, asparagine and aspartic acid, acetate, pyruvic acid, pyruvate, oxalate, malate, fumarate, lactate, lactic acid; citrate, bicarbonate, malonate, succinate, oxaloacetate, α-ketoglutarate, 3-hydroxybutyrate, isocitrate and urea.

Pyruvate is an endogenous compound that is very well tolerated by the human body, even in relatively high concentrations. As a precursor in the citric acid cycle, pyruvate plays an important metabolic role in the human body. Pyruvate is converted into different compounds: its transamination results in alanine, via oxidative decarboxylation; pyruvate is converted into acetyl-CoA and carbon dioxide (which is further converted to bicarbonate), the reduction of pyruvate results in lactate and its carboxylation in oxaloacetate.

Further, the metabolic conversion of hyperpolarized ¹³C-pyruvate into its metabolites hyperpolarized ¹³C-lactate, hyperpolarized ¹³C-bicarbonate (in the case of ¹³C₁-pyruvate, ¹³C_{1,2}-pyruvate or ¹³C_{1,2,3}-pyruvate only) and hyperpolarized ¹³C-alanine can be used to study metabolic processes in the human body using MR. ¹³C₁-pyruvate has a T₁ relaxation in human full blood at 37° C. of about 42 s, however, the conversion of hyperpolarized ¹³C-pyruvate to hyperpolarized ¹³C-lactate, hyperpolarized ¹³C-bicarbonate and hyperpolarized ¹³C-alanine has been found to be fast enough to allow signal detection from the ¹³C-pyruvate parent compound and its metabolites. The amount of alanine, bicarbonate and lactate is dependent on the metabolic status of the tissue under investigation. The MR signal intensity of hyperpolarized ¹³C-lactate, hyperpolarized ¹³C-bicarbonate and hyperpolarized ¹³C-alanine is related to the amount of these compounds and the degree of polarization left at the time of detection, hence by monitoring the conversion of hyperpolarized ¹³C-pyruvate to hyperpolarized ¹³C-lactate, hyperpolarized ¹³C-bicarbonate and hyperpolarized ¹³C-alanine it is possible to study metabolic processes in vivo in the human or non-human animal body by using non-invasive MRI, MRS, or MRSI.

It has been found that the MR signal amplitudes arising from the different pyruvate metabolites varies depending on the tissue type. The unique metabolic peak pattern formed by alanine, lactate, bicarbonate and pyruvate can be used as fingerprint for the metabolic state of the tissue under examination and thus allows for the discrimination between healthy tissue and unhealthy tissue. The use of hyperpolarized ¹³C-pyruvate for tumour imaging—with tumour tissue showing high metabolic activity—has been described in detail. In WO-A-2006/011810. Further, the use of hyperpolarized ¹³C-pyruvate for cardiac imaging has been described in WO-A-2006/054903.

Thus, in a preferred embodiment, embodiments of the invention provides a method of ¹³C-MR imaging and/or ¹³C-MR spectroscopy and/or ¹³C-MR spectroscopy imaging for detecting inflammation or infection using an imaging medium comprising hyperpolarized ¹³C-pyruvate.

Embodiments of the invention may solve the problem of how to detect sites of inflammation or infection. This is particularly important for occult infections, which are difficult to diagnose and detect. By the method of embodiments of the invention the anatomical location of diseased areas is identified. Further, by the method of embodiments of the invention a site of inflammation or infection may be quantified and information about the metabolic process of the disease activity may be provided. Hence, the method involves the benefits of anatomic imaging plus the addition of being able to characterise metabolic processes. Detecting the alterations of molecular processes may be more sensitive and specific than an anatomical description of disease. The hyperpolarized carbon-13 MRSI used in the method of embodiments of the invention dramatically increases the sensitivity for molecular processes. The subjective and quantitative imaging method of embodiments of the invention may detect disease earlier and may also better tailor therapy. This could be particularly important in the treatment of diseases with an inflammatory component such as asthma, chronic bronchitis, COPD, and multiple sclerosis where choice of medication is difficult and progression of disease difficult to monitor. In addition, embodiments of the invention may also help accelerate drug development since smaller numbers of subjects and shorter amounts of time are needed when the non-invasive method of embodiments of the invention is available to measure disease activity.

As an application of the art, we have shown that ¹³C-pyruvate can be used to detect inflammation. However, potentially any substance created with an isotope that may be hyperpolarized may be a candidate for detecting and monitoring inflammation or infection. Other substances that are candidates for detecting inflammation or infection with the hyperpolarized MRI technique include substances containing isotopes of oxygen, nitrogen, xenon, helium, and fluorine.

The term “¹³C-pyruvate” denotes a salt of ¹³C-pyruvic acid. In the following the terms pyruvate, ¹³C-pyruvate and ¹³C₁-pyruvate are used interchangeably and all denote ¹³C₁-pyruvate. Likewise the terms pyruvic acid, ¹³C-pyruvic acid and ¹³C₁-pyruvic acid are used interchangeably and all denote ¹³C₁-pyruvic acid. Further, the terms lactate, ¹³C-lactate and ¹³C₁-lactate are used interchangeably and all denote ¹³C₁-lactate, unless further specified.

The terms “hyperpolarized” and “polarised” are used interchangeably hereinafter and denote a nuclear polarization level in excess of 0.1%, more preferred in excess of 1% and most preferred in excess of 10%.

The level of polarization may for instance be determined by solid state ¹³C-NMR measurements in solid hyperpolarized ¹³C-pyruvate, e.g. solid hyperpolarized ¹³C pyruvate obtained by dynamic nuclear polarization (DNP) of ¹³C-pyruvate. The solid state ¹³C-NMR measurement preferably consists of a simple pulse-acquire NMR sequence using a low flip angle. The signal intensity of the hyperpolarized ¹³C-pyruvate in the NMR spectrum is compared with signal intensity of ¹³C-pyruvate in a NMR spectrum acquired before the polarization process. The level of polarization is then calculated from the ratio of the signal intensities of before and after polarization.

In a similar way, the level of polarization for dissolved hyperpolarized ¹³C pyruvate may be determined by liquid state NMR measurements. Again the signal intensity of the dissolved hyperpolarized ¹³C-pyruvate is compared with the signal intensity of the dissolved ¹³C-pyruvate before polarization. The level of polarization is then calculated from the ratio of the signal intensities of ¹³C-pyruvate before and after polarization.

The term “imaging medium” denotes a liquid composition comprising but not limited to a hyperpolarized ¹³C-substance, such as hyperpolarized ¹³C-pyruvate, as the MR active agent. The imaging medium according to embodiments of the invention may be used as imaging medium in MR imaging or as MR spectroscopy agent in MR spectroscopy and MR spectroscopic imaging.

The imaging medium according to the method of embodiments of the invention may be used as imaging medium for in vivo MR imaging, spectroscopy and/or spectroscopic imaging, i.e. MR imaging, spectroscopy and/or spectroscopic imaging carried out on living human or non-human animal beings. Further, the imaging medium according to the method of embodiments of the invention may be used as imaging medium for in vitro MR imaging, spectroscopy and/or spectroscopic imaging, e.g. for detecting and monitoring of inflammation or infection in cell cultures or ex vivo tissues. Cell cultures may be derived from cells obtained from samples derived from the human or non-human animal body like for instance blood, urine or saliva while ex vivo tissue may be obtained from biopsies or surgical procedures.

The isotopic enrichment of the hyperpolarized ¹³C-pyruvate used in the method of embodiments of the invention is preferably at least 75%, more preferably at least 80% and especially preferably at least 90%, an isotopic enrichment of over 90% being most preferred. Ideally, the enrichment is 100%. ¹³C-pyruvate used in the method of embodiments of the invention may be isotopically enriched at the C1-position (in the following denoted ¹³C₁-pyruvate), at the C2-position (in the following denoted ¹³C₂-pyruvate), at the C3-position (in the following denoted ¹³C₃-pyruvate), at the C₁- and the C2-position (in the following denoted ¹³C_{1,2}-pyruvate), at the C1- and the C3-position (in the following denoted ¹³C_{1,3}-pyruvate), at the C2- and the C3-position (in the following denoted ¹³C_{2,3}-pyruvate) or at the C1-, C2- and C3-position (in the following denoted ¹³C_{1,2,3}-pyruvate). Isotopic enrichment at the C1-position is preferred since ¹³C₁-pyruvate has a higher T₁ relaxation in human hill blood at 37° C. (about 42 s) than ¹³C-pyruvate which is isotopically enriched at other C-positions.

Hyperpolarization of NMR active ¹³C-nuclei may be achieved by different methods which are for instance described in described in WO-A-98/30918, WO-A-99/24080 and WO-A-99/35508, which are incorporated herein by reference and hyperpolarization methods are polarization transfer from a noble gas, “brute force”, spin refrigeration, the parahydrogen method and dynamic nuclear polarization (DNP).

To obtain hyperpolarized ¹³C-pyruvate, it is preferred to either polarise ¹³C pyruvate directly or to polarise ¹³C-pyruvic acid and convert the polarised ¹³C-pyruvic acid to polarised ¹³C-pyruvate, e.g. by neutralisation with a base.

One suitable way for obtaining hyperpolarized ¹³C-pyruvate is the polarization transfer from a hyperpolarized noble gas which is described in WO-A-98/30918. Noble gases having non-zero nuclear spin can be hyperpolarized by the use of circularly polarised light. A hyperpolarized noble gas, preferably He or Xe, or a mixture of such gases, may be used to effect hyperpolarization of ¹³C-nuclei. The hyperpolarized gas may be in the gas phase, it may be dissolved in a liquid/solvent, or the hyperpolarized gas itself may serve as a solvent. Alternatively, the gas may be condensed onto a cooled solid surface and used in this form, or allowed to sublime. Intimate mixing of the hyperpolarized gas with ¹³C-pyruvate or ¹³C-pyruvic acid is preferred. Hence, if ¹³C-pyruvic acid is polarised, which is a liquid at room temperature, the hyperpolarized gas is preferably dissolved in a liquid/solvent or serves as a solvent. If ¹³C pyruvate is polarised, the hyperpolarized gas is preferably dissolved in a liquid/solvent, which also dissolves pyruvate.

Another suitable way for obtaining hyperpolarized ¹³C-pyruvate is that polarization is imparted to ¹³C-nuclei by thermodynamic equilibration at a very low temperature and high field. Hyperpolarization compared to the operating field and temperature of the NMR spectrometer is effected by use of a very high field and very low temperature (brute force). The magnetic field strength used should be as high as possible, suitably higher than 1 T, preferably higher than 5 T, more preferably 15 T or more and especially preferably 20 T or more. The temperature should be very low, e.g. 4.2 K or less, preferably 1.5 K or less, more preferably 1.0 K or less, especially preferably 100 mK or less.

Another suitable way for obtaining hyperpolarized ¹³C-pyruvate is the spin refrigeration method. This method covers spin polarization of a solid compound or system by spin refrigeration polarization. The system is doped with or intimately mixed with suitable crystalline paramagnetic materials such as Ni²⁺, lanthanide or actinide ions with a symmetry axis of order three or more. The instrumentation is simpler than required for DNP with no need for a uniform magnetic field since no resonance excitation field is applied. The process is carried out by physically rotating the sample around an axis perpendicular to the direction of the magnetic field. The pre-requisite for this method is that the paramagnetic species has a highly anisotropic g-factor. As a result of the sample rotation, the electron paramagnetic resonance will be brought in contact with the nuclear spins, leading to a decrease in the nuclear spin temperature. Sample rotation is carried out until the nuclear spin polarization has reached a new equilibrium.

In a preferred embodiment, dynamic nuclear polarization (DNP) is used to obtain hyperpolarized ¹³C-pyruvate. In DNP, polarization of MR active nuclei in a compound to be polarized is affected by a polarization agent or so-called DNP agent, a compound comprising unpaired electrons. During the DNP process, energy, normally in the form of microwave radiation, is provided, which will initially excite the DNP agent. Upon decay to the ground state, there is a transfer of polarization from the unpaired electron of the DNP agent to the NMR active nuclei of the compound to be polarised, e.g. to the ¹³C nuclei in ¹³C-pyruvate. Generally, a moderate or high magnetic field and a very low temperature are used in the DNP process, e.g. by carrying out the DNP process in liquid helium and a magnetic field of about 1 T or above. Alternatively, a moderate magnetic field and any temperature at which sufficient polarization enhancement is achieved may be employed. The DNP technique is for example further described in WO-A-98/58272 and in WO-A-01/96895, both of which are included by reference herein.

To polarise a compound by the DNP method, a mixture of the compound to be polarised and a. DNP agent is prepared (“a sample”) which is then frozen and inserted into a DNP polariser for polarization. After the polarization, the frozen solid hyperpolarized sample is rapidly transferred into the liquid state either by melting it or by dissolving it in a suitable dissolution medium. Dissolution is preferred and the dissolution process of a frozen hyperpolarized sample and suitable devices therefore are described in detail in WO-A-02/37132. The melting process and suitable devices for the melting are for instance described in WO-A-02/36005.

In order to obtain a high polarization level in the compound to be polarised said compound and the DNP agent need to be in intimate contact during the DNP process. This is not the case if the sample crystallizes upon being frozen or cooled. To avoid crystallization, either glass formers need to be present in the sample or compounds need to be chosen for polarization which do not crystallize upon being frozen but rather form a glass.

As mentioned earlier ¹³C-pyruvic acid or ¹³C-pyruvate is suitable starting materials to obtain hyperpolarized ¹³C-pyruvate.

Isotopically enriched ¹³C-pyruvate is commercially available, e.g. as sodium ¹³C-pyruvate. Alternatively, it may be synthesized as described by S. Anker, J. Biol. Chem. 176, 1948, 133-1335.

Several methods for the synthesis of ¹³C₁-pyruvic acid are known in the art. Briefly, Seebach et al., Journal of Organic Chemistry 40(2), 1975, 231-237 describe a synthetic route that relies on the protection and activation of a carbonyl-containing starting material as an S,S-acetal, e.g. 1,3-dithian or 2-methyl-1,3-dithian. The dithiane is metallated and reacted with a methyl-containing compound and/or ¹³CO₂. By using the appropriate isotopically enriched ¹³C-component as outlined in this reference, it is possible to obtain ¹³C₁-pyruvate, ¹³C₂-pyruvate or ¹³C_{1,2}-pyruvate. The carbonyl function is subsequently liberated by use of conventional methods described in the literature. A different synthetic route starts from acetic acid, which is first converted into acetyl bromide and then reacted with Cu¹³CN. The nitrile obtained is converted into pyruvic acid via the amide (see for instance S. H. Anker et al., J. Biol. Chem. 176 (1948), 1333 or J. E. Thirkettle, Chem Commun. (1997), 1025). Further, ¹³C-pyruvic acid may be obtained by protonating commercially available sodium ¹³C-pyruvate, e.g. by the method described in U.S. Pat. No. 6,232,497 or by the method described in WO-A-2006/038811.

The hyperpolarization of ¹³C-pyruvic acid by DNP is described in detail in WO-A1-2006/011809, which is incorporated herein by reference. Briefly, ¹³C-pyruvic acid may be directly used for DNP since it forms a glass when frozen. After DNP, the frozen hyperpolarized ¹³C-pyruvic acid needs to be dissolved and neutralised, i.e. converted to ¹³C-pyruvate. For the conversion, a strong base is needed. Further, since ¹³C-pyruvic acid is a strong acid, a DNP agent needs to be chosen which is stable in this strong acid. A preferred base is sodium hydroxide and conversion of hyperpolarized ¹³C-pyruvic acid with sodium hydroxide results in hyperpolarized sodium ¹³C-pyruvate, which is the preferred ¹³C-pyruvate for an imaging medium which is used for in vivo MR imaging, spectroscopy, and/or spectroscopic imaging, i.e. MR imaging, spectroscopy, and/or spectroscopic imaging carried out on living human or non-human animal beings.

Alternatively, ¹³C-pyruvate, i.e. a salt of ¹³C-pyruvic acid can be used for DNP. Preferred salts are those ¹³C-pyruvates which comprise an inorganic cation from the group consisting of NH₄⁺, K⁺, Rb⁺, Cs⁺, Ca²⁺, Sr²⁺ and Ba²⁺, preferably NH₄⁺, K⁺, Rb⁺ or Cs⁺, more preferably K⁺, Rb⁺, Cs⁺ and most preferably Cs⁺, as in detail described in WO-A-2007/111515 and incorporated by reference herein. The synthesis of these preferred ¹³C-pyruvates is disclosed in WO-A-2007/111515 as well. If the hyperpolarized ¹³C-pyruvate is used in an imaging medium for in vivo MR imaging and/or spectroscopy it is preferred to exchange the inorganic cation from the group consisting of NH₄⁺, K⁺, Rb⁺, Cs⁺, Ca²⁺, Sr²⁺ and Ba²⁺ by a physiologically very well tolerable cation like Na⁺ or meglumine. This may be done by methods known in the art like the use of a cation exchange column.

Further preferred salts are 13C-pyruvates of an organic amine or amino compound, preferably TRIS-¹³C₁-pyruvate or meglumine-¹³C₁-pyruvate, as in detail described in WO-A2-2007/069909 and incorporated by reference herein. The synthesis of these preferred ¹³C-pyruvates is disclosed in WO-A2-2007/069909 as well.

If the hyperpolarized ¹³C-pyruvate used in the method of embodiments of the invention is obtained by DNP, the sample to be polarised comprising ¹³C-pyruvic acid or ¹³C-pyruvate and a DNP agent may further comprise a paramagnetic metal ion. The presence of paramagnetic metal ions in composition to be polarised by DNP has found to result in increased polarization levels in the ¹³C-pyruvic acid/¹³C-pyruvate as described in detail in WO-A2-2007/064226, which is incorporated herein by reference.

As mentioned earlier, the imaging medium according to the method of embodiments of the invention may be used as imaging medium for in vivo MR imaging, spectroscopy, and/or spectroscopic imaging, i.e. MR imaging, spectroscopy, and/or spectroscopic imaging carried out on living human or non-human animal beings. Such an imaging medium preferably comprises in addition to the MR active agent ¹³C-substance, such as ¹³C-pyruvate, an aqueous carrier, preferably a physiologically tolerable and pharmaceutically accepted aqueous carrier like water/saline, a buffer or a mixture of buffers. The imaging medium may further comprise conventional pharmaceutically acceptable carriers, excipients and formulation aids. Thus, the imaging medium may for example include stabilizers, osmolality adjusting agents, solubilising agents and the like, e.g. formulation aids such as are conventional for diagnostic compositions in human or veterinary medicine.

Further, the imaging medium according to the method of embodiments of the invention may be used as imaging medium for in vitro MR imaging, spectroscopy, and/or spectroscopic imaging, e.g. for detecting inflammation or infection in cell cultures or ex vivo tissues. Such an imaging medium preferably comprises in addition to the MR active agent ¹³C-substance, such as ¹³C-pyruvate, a solvent which is compatible with and used for in vitro cell or tissue assays, for instance DMSO or methanol or solvent mixtures comprising an aqueous carrier and a non aqueous solvent, for instance mixtures of DMSO and water or a buffer solution or methanol and water or a buffer solution. As it is apparent for the skilled person, pharmaceutically acceptable carriers, excipients and formulation aids may be present in such an imaging medium but are not required for such a purpose.

If the hyperpolarized ¹³C-pyruvate is used as an imaging agent for the detection of infection in an in vitro method of MR imaging or spectroscopy, e.g. using cell cultures or ex vivo tissue, the imaging medium comprising the hyperpolarized ¹³C-pyruvate that is added to the cell culture or ex vivo tissue is 10 mM to 100 mM in ¹³C-pyruvate, more preferably 20 mM to 90 mM and most preferably 40 to 80 mM in ¹³C-pyruvate.

Furthermore, the types of inflammatory and infectious diseases detected by the method of embodiments of the invention may vary. The method may be used to detect a range of diseases where the immune system is activated or altered. These diseases may affect any body tissue such as the skin and skeletal, digestive, muscular, lymphatic, endocrine, nervous, cardiovascular, male or female reproductive, and urinary systems. The method may detect autoimmune disease to any part of the body. A non-comprehensive list of clinical diseases with an autoimmune component include rheumatoid arthritis, juvenile idiopathic arthritis, systemic lupus erythematosus, scleroderma, dermatomyositis, myocarditis, Crohns and multiple sclerosis. This method may be used to detect the inflammatory response to healing after trauma. This method may be used to detect chronic diseases that have a component of inflammation such as artherosclerosis, osteoarthritis, tendinitis, bursitis, gouty arthritis, COPD, asthma, and chronic bronchitis. This method may detect inflammation in response to infections (e.g. bacterial, viral, fungal, parasitic, or other infectious source) of any part of the body including the skin, extremities, muscles, connective tissues, bones, joints, nervous system, and internal organs of the head, neck, chest, and abdomen. Inflammation plays a large role in transplantation. The method may detect alterations in the immune system in the setting of transplantation such as acute and chronic transplant rejection of solid organs, post-transplant lymphoproliferative disease and graft-versus host disease.

The method of embodiments of the invention includes detection of all these types of conditions mentioned above. A preferred embodiment is a method of 13C-MR imaging, 13C-MR spectroscopy, and/or 13C-MR spectroscopic imaging for detecting arthritis, and more preferably rheumatoid arthritis, wherein an imaging medium comprising a hyperpolarized ¹³C-substance, preferably hyperpolarized ¹³C-pyruvate, is used.

In another embodiment, the imaging medium further comprises lactate. Hence the imaging medium according to the method of embodiments of the invention comprises non-hyperpolarized lactate, hereinafter denoted lactate, in addition to hyperpolarized ¹³C-pyruvate. Suitably, lactate is added in the form of lactic acid or a salt of lactic acid, preferably lithium lactate or sodium lactate, most preferably sodium lactate. Imaging media comprising lactate and hyperpolarized ¹³C-pyruvate, and method for using such, is further described in WO2008/020765 which is incorporated herein by reference.

Inflammation and infection can be detected by the method of embodiments of the invention by following the ¹³C-pyruvate signal and the signal of its metabolite ¹³C-lactate over time in viable, e.g. non inflammatory cells, the ¹³C-pyruvate signal decays over time. The ¹³C-lactate signal increases first due to metabolic conversion of ¹³C-pyruvate to ¹³C-lactate and then slowly decreases mainly due to relaxation. In areas of inflammation, the metabolism of pyruvate is upregulated and the conversion of ¹³C-pyruvate to ¹³C-lactate is increased. With the use of an imaging medium comprising hyperpolarized ¹³C-pyruvate, this higher metabolic activity can be seen by an increased production of ¹³C-lactate which can be detected by ¹³C-MR detection.

It has further been found that the addition of lactate—either being present in the imaging medium according to embodiments of the invention or being added/administered separately—leads to an increased amount of observable ¹³C-lactate and thus an increased MR signal from ¹³C-lactate.

The term “¹³C-MR detection” denotes ¹³C-MR imaging or ¹³C-MR spectroscopy or combined ¹³C-MR imaging and ¹³C-MR spectroscopy, i.e. ¹³C-MR spectroscopic imaging. The term further denotes ¹³C-MR spectroscopic imaging at various time points.

An MR imaging sequence is applied that encodes the volume of interest in a combined frequency and spatially selective way and the ¹³C-MR signal of ¹³C-pyruvate is followed by MR imaging or spectroscopic imaging over a time period from the addition of the imaging agent (t=0) to about 1 min or until the ¹³C-MR signal undetectable due to the signal decay via T₁ relaxation. In the same time period, the appearance, increase and/or decrease of the ¹³C-lactate signal is monitored. To get a quantitative assessment, MR imaging, spectroscopy, or spectroscopic imaging of healthy cells or tissue may carried out and the results—i.e. the amount or rate of ¹³C-lactate formed over a given time period—may be compared.

If the hyperpolarized ¹³C-pyruvate is used as an imaging agent for the detection of inflammation or infection in an in vivo method of MR imaging, spectroscopy or spectroscopic imaging, e.g. in a living human or non-human animal body, the imaging medium containing the hyperpolarized ¹³C-pyruvate is preferably administered to said body parenterally, preferably intravenously. Generally, the body under examination is positioned in the MR magnet. Dedicated ¹³C-MR RF-coils are positioned to cover the area of interest. Dosage and concentration of the imaging medium will depend upon a range of factors such as toxicity and the administration route. Generally, the imaging medium is administered in a concentration of up to 1 mmol ¹³C-pyruvate per kg bodyweight, preferably 0.01 to 0.5 mmol/kg, more preferably 0.1 to 0.3 mmol/kg. The administration rate is preferably less than 10 ml/s, more preferably less than 6 ml/s and most preferable of from 5 ml/s to 0.1 ml/s. At less than 400 s after the administration, preferably less than 120 s, more preferably less than 60 s after the administration, especially preferably 20 to 50 s an MR imaging sequence is applied that encodes the volume of interest in a combined frequency and spatial selective way. This will result in metabolic images of ¹³C-pyruvate, ¹³C-lactate and/or other ¹³C-labeled metabolic products. The exact time of applying an MR sequence is highly dependent on the volume of interest for detecting infection or inflammation.

The encoding of the volume of interest can be achieved by using so-called spectroscopic imaging sequences, such as but not limited to those described in for instance T. R. Brown et al., Proc Natl Acad Sci USA 79, 3523-3526 (1982); A. A. Maudsley et al., J Magn Res 51, 147-152 (1983); D. Mayer et al., Magn Reson Med 56, 932-937 (2006); S. J. Kohler et al., Magn Reson Med 58(1), 65-9 (2007); Y-F. Yen et al., Magn Reson Med (Epub ahead of print) Mar. 24 (2009). Spectroscopic image data contain a number of volume elements in which each element contains a full 13C-MR spectrum. 13C-pyruvate and its metabolite 13C-lactate have their unique position in a 13C-MR spectrum and their resonance frequency can be used to identify them. The integral of the spectral peak at its resonance frequency is directly related to the amount of 13C-pyruvate and 13C-lactate, respectively. When the amount of 13C-pyruvate and 13C-lactate is estimated using the spectral peak integral analysis or time domain fitting routines as described for instance in L. Vanhamme et al., J Magn Reson 129, 35-43 (1997), or least-squares chemical shift separation method as described for example in S. B. Reeder et al., J Magn Reson Imaging 26, 1145-1152 (2007) and Y. S. Levin et al., Magn Reson Med. 58(2), 245-52 (2007), images can be generated for 13C-pyruvate and 13C-lactate in which a colour coding or grey coding is representative for the amount of 13C-pyruvate and 13C-lactate measured.

Although spectroscopic imaging methods have proven their value in producing metabolic images using all kinds of MR nuclei e.g. ¹H, ³¹P, ²³Na, the amount of repetitions needed to fully encode the spectroscopic image makes this approach less suitable for hyperpolarized ¹³C. Care has to be taken to ensure hyperpolarized ¹³C-signal is available during the whole MR data acquisition. This can be achieved by reducing the RF-pulse excitation flip angles or by applying variable flip angles as described for example in L. Zhao et al., J Magn Reson, B(113), 179-183 (1996), or by multi-band RF excitation designs as described for example in P. E. Z. Larson et al., J Magn Reson 194: 121-127 (2008), that is applied in every phase encoding step. Higher matrix sizes require more phase encoding steps and longer scan times.

Imaging methods based on the pioneering work of P. C. Lauterbur (Nature, 242, 190-191, (1973) and P. Mansfield (J. Phys. C. 6, L422-L426 (1973)), which apply a readout gradient during the data acquisition, will allow for higher signal to noise images or the equivalent, higher spatial resolution images. However, these imaging methods in their basic form will not be able to produce separate images for ¹³C-pyruvate and ¹³C-lactate, i.e. the identification of specific metabolites is not possible.

In another embodiment, imaging sequences are used that will make use of multi-echoes to code for the frequency information. Sequences that can produce separate water and fat ¹H-images are for example described in G. Glover, J Magn Reson Imaging1, 521-530 (1991) and S. B. Reeder et al., Magn Reson Med 51, 35-45 (2004). Since the metabolites to be detected and as such their MR frequencies are known, the approach discussed in the references above can be applied to acquire direct images of ¹³C-pyruvate and ¹³C-lactate. This procedure makes more efficient use of the hyperpolarized ¹³C-MR signal, giving a better signal quality compared to spectroscopic imaging, a higher spatial resolution and faster acquisition times.

In a preferred embodiment, the method according to embodiments of the invention comprises acquiring direct ¹³C-MR images or spectra of ¹³C-pyruvate and ¹³C-lactate from a human or non-human animal body pre-administered with an imaging medium comprising hyperpolarized ¹³C-pyruvate or from a cell culture or ex vivo tissue the imaging medium has been added to. In the method described, infection or inflammation is identified and detected by high ¹³C-signal intensity from ¹³C-lactate or an increased rate of formation of ¹³C-lactate. Hyperpolarized ¹³C-pyruvate imaging according to embodiments of the invention shows increased metabolism to lactate in inflammation and infection.

To correct for the pyruvate signal, both lactate and pyruvate images may be normalized to the maximum value in each individual image. Second, the normalized lactate image is multiplied by the inverted pyruvate image, e.g. the maximum pyruvate signal in the image minus the pyruvate level for every pixel. As a last step, the intermediate result gained in the operation above is multiplied by the original lactate image. Alternatively, the pyruvate and lactate peak intensities in each pixel of their respective images can be it to a kinetic model of the flux of ¹³C-label between pyruvate and lactate to obtain rate constants for label flux and the spin lattice relaxation times. Correction may need to be made for the effect of multiple RF pulses on the loss of polarization.

Anatomical and/or perfusion information may be included in the detection of inflammation or infection according to the method of embodiments of the invention, if the method is used for detection of inflammation or infection in vivo. Anatomical information may for instance be obtained by acquiring proton MR images with or without employing a suitable contrast agent. Relative perfusion can be determined by using an MR contrast agent like for instance Omniscan™. Likewise, MR imaging techniques for perfusion measurement without the administration of a contrast agent are known in the art. In a preferred embodiment, a non-metabolised hyperpolarized ¹³C-contrast agent is used to determine quantitative perfusion. Suitable techniques and contrast agents are for instance described in WO-A-02/23209. In a more preferred embodiment, hyperpolarized ¹³C-pyruvate is used to determine quantitative perfusion.

In another preferred embodiment, the imaging medium comprising hyperpolarized ¹³C-pyruvate is administered repeatedly, thus allowing longitudinal studies. Due to the low toxicity of pyruvate and its favourable safety profile, repeated doses of this compound are well tolerated by the patient.

The results obtained in the method of embodiments of the invention for instance may allow the physician to choose the appropriate treatment for the patient under examination. In a further preferred embodiment, the method of embodiments of the invention may be used to determine whether treatment is successful.

Viewed from a further aspect, embodiments of the invention provides the use of a hyperpolarized ¹³C-substance for the manufacture of an imaging medium for use in a method of ¹³C-MR imaging, ¹³C-MR spectroscopy and/or ¹³C-MR spectroscopic imaging for detecting inflammation or infection. More preferably, embodiments of the invention provides the use of hyperpolarized ¹³C-pyruvate for the manufacture of an imaging medium for use in a method of ¹³C-MR imaging, ¹³C-MR spectroscopy and/or ¹³C-MR spectroscopic imaging for detecting inflammation or infection. Preferably, the hyperpolarized ¹³C-pyruvate used for the manufacture of the imaging medium is obtained by dynamic nuclear polarization of ¹³C-pyruvic acid or ¹³C-pyruvate. Optionally, lactate may be added to ¹³C-substance for the manufacture of the imaging medium.

The manufacture and preferred embodiments of the manufacture of hyperpolarized ¹³C-pyruvate from ¹³C-pyruvic acid or ¹³C-pyruvate as well as the manufacture of an imaging medium comprising hyperpolarized ¹³C and optionally lactate is described in detail elsewhere in this application.

In a preferred embodiment, embodiments of the invention provides the use of hyperpolarized ¹³C-pyruvate and optionally lactate for the manufacture of an imaging medium for use in a method of ¹³C-MR imaging, ¹³C-MR spectroscopy and/or ¹³C-MR spectroscopic imaging for detecting inflammation or infection by acquiring direct ¹³C images and/or ¹³C-spectra of ¹³C-pyruvate and ¹³C-lactate from a human or non-human animal body which has been pre-administered with the imaging medium or from a cell culture or ex vivo tissue to which the imaging medium has been added to.

In another preferred embodiment the invention provides use of an imaging medium comprising a hyperpolarized ¹³C-substance in a method of ¹³C-MR imaging, ¹³C-MR spectroscopy and/or ¹³C-MR spectroscopic imaging for detecting inflammation or infection in a human on non-human animal body. The imaging medium has preferably been preadministered to the human or non-human animal body.

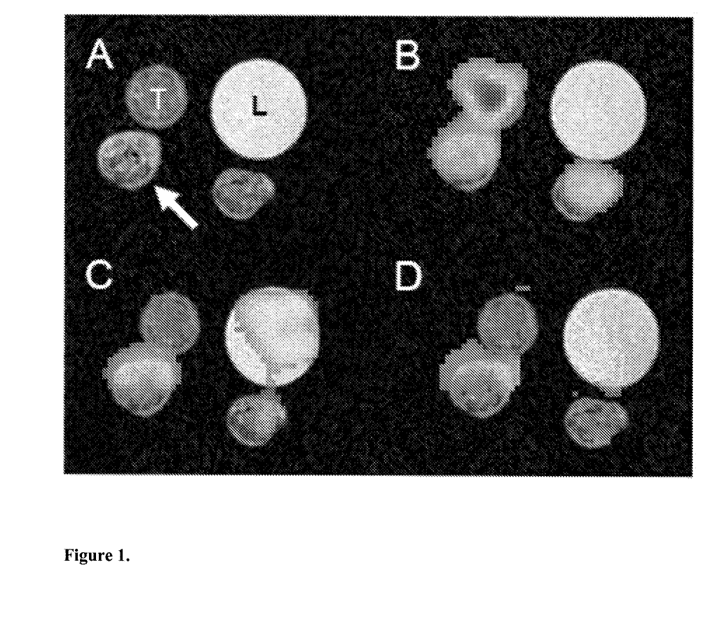

FIG. 1 shows metabolic maps of arthritic joints. At 20 sec after injection of hyperpolarized [1-¹³C]pyruvate the maps demonstrate increased lactate production in the arthritic paw. A: T2-weighted anatomic image shows tissue swelling at the arthritic right hind paw (arrow) in comparison to the normal left paw and is overlayed on the subsequent metabolic maps with tail (T) and non-polarized ¹³C-lactate (L) reference tube. Maps show B: [1-¹³C]pyruvate, C: [1-¹³C]lactate, and D: the ratio of [1-¹³C]lactate/[1-13C]pyruvate.

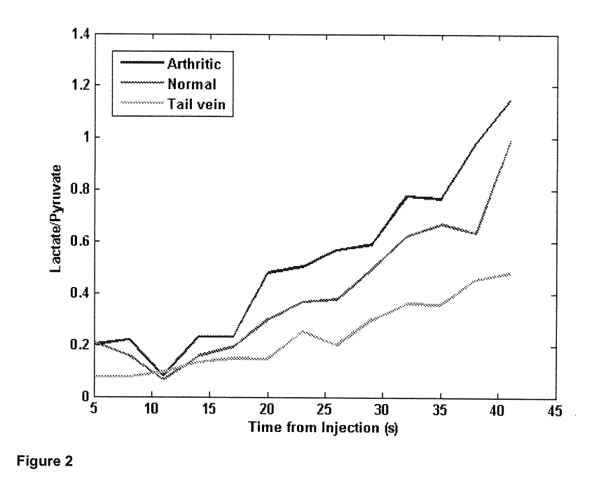

FIG. 2 shows time resolved imaging wherein the increased production of [1-¹³C]lactate in the arthritic paw in one rat (blue) is in comparison to the normal paw (right) and tail (green).

In order to promote a further understanding of the invention, the following examples are provided. These examples are illustrative, and should not be construed to be any sort of limitation on the scope of the invention.

### EXAMPLES

### Example 1

### Detection of Arthritis

Arthritis was induced in six juvenile Sprague Dawley rats (age 4-5 weeks, mean weight 114 grams) with injection of 0.4 μL/g complete Freund's adjuvant (3 rats at the right knee and 3 rats at the right ankle). Arthritic joints were imaged 7 days after induction with ¹³C MRS on a GE 3 T scanner equipped with self-shielded gradients (40 mT/m, 150 mT/m/ms) and a custom-built dual-tuned (¹H/¹³C) quadrature coil (Ø=80 mm) for both excitation and signal reception. 0.5 mL of a 100 mM solution of ¹³C-1-pyruvate was hyperpolarized by DNP (15-20% liquid state polarization) and injected via the tail vein. Single-time point MRS analysis of 13C-1 pyruvate and its metabolites was obtained 20 sec after injection with a FID CSI sequence (voxel=2.5×2.5×10 mm, FOV=4×4 cm). Time resolved imaging was obtained with a 1D EPSI sequence during a second hyperpolarized ¹³C-pyruvate injection. Mean signal intensities of pyruvate and lactate were obtained with ROI analysis at the joints and normal and arthritic joints were compared with the T-test.

Arthritic joints were found to be erythematous and swollen (mean±SD=0.5±0.2 mm greater in thickness), had a histological score of 3/4 for inflammation (compared with 0/4 at the normal joint), and showed T2-weighted changes of inflammation on the anatomic MR images. [1-¹³C]pyruvate and metabolized [1-¹³C]lactate appeared increased at the arthritic joints on the FID CSI images (FIG. 1A, B) and tended towards significant difference by ROI analysis of the ratio of metabolite at the joint to total ¹³C [pyruvate arthritic=0.34 vs. normal=28, p<0.17; lactate arthritic=0.21 vs. normal=0.16, p<0.12]. Although increased blood flow in inflamed tissue may account for the increased delivery of imaging agent, the rate of conversion to lactate was also increased in the arthritic joints as shown by time resolved imaging (FIG. 2) and by the ratio of lactate to total ¹³C (arthritic=0.62 vs. normal=0.56, p<0.03).

Hence, according to these results hyperpolarized [1-¹³C]pyruvate imaging shows increased metabolism to lactate in joints affected by arthritis. Increased lactate production may serve as a marker of arthritis activity.

As used herein, approximating language may be applied to modify any quantitative representation that may vary without resulting in a change in the basic function to which it is related. Accordingly, a value modified by a term or terms, such as “about” and “substantially,” may not be limited to the precise value specified, in some cases. The modifier “about” used in connection with a quantity is inclusive of the stated value and has the meaning dictated by the context (for example, includes the degree of error associated with the measurement of the particular quantity). “Optional” or “optionally” means that the subsequently described event or circumstance may or may not occur, or that the subsequently identified material may or may not be present, and that the description includes instances where the event or circumstance occurs or where the material is present, and instances where the event or circumstance does not occur or the material is not present. The singular forms “a”, “an” and “the” include plural referents unless the context clearly dictates otherwise. All ranges disclosed herein are inclusive of the recited endpoint and independently combinable.

As used herein, the phrases “adapted to,” “configured to,” and the like refer to elements that are sized, arranged or manufactured to form a specified structure or to achieve a specified result. While the invention has been described in detail in connection with only a limited number of embodiments, it should be readily understood that the invention is not limited to such disclosed embodiments. Rather, the invention can be modified to incorporate any number of variations, alterations, substitutions or equivalent arrangements not heretofore described, but which are commensurate with the spirit and scope of the invention. Additionally, while various embodiments of the invention have been described, it is to be understood that aspects of the invention may include only some of the described embodiments. Accordingly, the invention is not to be seen as limited by the foregoing description. It is also anticipated that advances in science and technology will make equivalents and substitutions possible that are not now contemplated by reason of the imprecision of language and these variations should also be construed where possible to be covered.

What is claimed as new and desired to be protected by Letters Patent of the United States is:

1. A method for detecting inflammation or infection by ¹³C-MR imaging, ¹³C-MR spectroscopy and/or ¹³C-MR spectroscopic imaging, wherein an imaging medium comprising hyperpolarized ¹³C-pyruvate is used, and wherein inflammation or infection are detected by high ¹³C-signal intensity from ¹³C-lactate or an increased rate of formation of ¹³C-lactate.

2. The method as claimed in claim 1 wherein the imaging medium is administered to a human or non-human animal body and said ¹³C-MR imaging, ¹³C-MR, spectroscopy and/or ¹³C-MR spectroscopic imaging is carried out for detecting inflammation or infection in said human or non-human animal body.

3. The method as claimed in claim 1 wherein the imaging medium is added to a cell culture or ex vivo tissue and said ¹³C-MR imaging and/or ¹³C-MR spectroscopy is carried out for detecting inflammation or infection in said cell culture or ex vivo tissue.

4. The method as claimed in claim 1 wherein ¹³C-signal intensities from ¹³C-pyruvate and its metabolite ¹³C-lactate are followed over time.

5. The method as claimed in claim 4 wherein the ¹³C-signal intensities from ¹³C-pyruvate and ¹³C-lactate are followed from the time point of the administration/addition of the imaging medium for about 1 minute, or until the ¹³C-MR signal is undetectable due to the signal decay via T₁ relaxation.

6. The method as claimed in claim 2 wherein to said human or non-human body lactate was administered prior to the administration/addition of said imaging medium.

7. The method as claimed in claim 3 wherein to said cell culture or ex vivo tissue lactate was added prior to the addition of said imaging medium.

8. The method as claimed in claim 1 wherein the hyperpolarized ¹³C-pyruvate is obtained by dynamic nuclear polarization of ¹³C-pyruvic acid or ¹³C-pyruvate.

9. Use of hyperpolarized ¹³C-pyruvate for the manufacture of an imaging medium for use in a method for detecting inflammation or infection by ¹³C-MR imaging, ¹³C-MR spectroscopy and/or ¹³C-MR spectroscopic imaging, wherein inflammation or infection are detected by high ¹³C-signal intensity from ¹³C-lactate or an increased rate of formation of ¹³C-lactate.

10. A method for detecting inflammation or infection in a human or non-human animal body by ¹³C-MR imaging, ¹³C-MR spectroscopy and/or ¹³C-MR spectroscopic imaging, wherein an imaging medium comprising hyperpolarized ¹³C-pyruvate has been preadministered to the human or non-human animal body, and wherein inflammation or infection are detected by high ¹³C-signal intensity from ¹³C-lactate or an increased rate of formation of ¹³C-lactate.

11. Use of an imaging medium comprising hyperpolarized ¹³C-pyruvate in a method for detecting inflammation or infection in a human on non-human animal body of ¹³C-MR imaging, ¹³C-MR spectroscopy and/or ¹³C-MR spectroscopic imaging, wherein inflammation or infection are detected by high ¹³C-signal intensity from ¹³C-lactate or an increased rate of formation of ¹³C-lactate.
